(19) United States
(12) Patent Application Publication
(10) Pub. No.: US 2025/0366807 A1
(43) Pub. Date: Dec. 4, 2025

(54) DIGITAL BREAST TOMOSYNTHESIS SYSTEM, METHOD, APPARATUS, AND STORAGE MEDIUM BASED ON X-RAY ARRAY SOURCE

(71) Applicant: Xi’an Jiaotong University, Xi’an (CN)

(72) Inventors: Xuanqin MOU, Xi’an (CN); Jiayu DUAN, Xi’an (CN); Yang LI, Xi’an (CN)

(73) Assignee: Xi’an Jiaotong University, Xi’an (CN)

(21) Appl. No.: 19/304,854

(22) Filed: Aug. 20, 2025

Related U.S. Application Data
(63) Continuation of application No. PCT/CN2023/081314, filed on Mar. 14, 2023.

(51) Int. Cl. A61B 6/50; A61B 6/02; A61B 6/03; A61B 6/04; A61B 6/06; A61B 6/40; A61B 6/42; A61B 6/58

(52) U.S. Cl. CPC A61B 6/502; A61B 6/025; A61B 6/032; A61B 6/0414; A61B 6/0435; A61B 6/06; A61B 6/4085; A61B 6/4283; A61B 6/584

(57) ABSTRACT

A digital breast tomosynthesis system includes a power supply module, an X-ray array source connected to the power supply module, a detection module, a data acquisition module, and an image reconstruction module. The detection module includes a detection platform, a detector, and a connecting arm; the plane X-ray array source is disposed on an opposite side of the detection platform. The flat-panel X-ray array source includes at least two ray source units, and a single X-ray array source is required for the arc-shaped plane X-ray array source. The system provided by the present disclosure can realize virtual rotational projection of an imaging object without moving parts, and obtain more longitudinal projection information through the plane X-ray array source, thereby improving the longitudinal resolution of reconstructed images.

### CROSS-REFERENCE TO RELATED APPLICATIONS

This application is a continuation of International Application No. PCT/CN2023/081314, filed on Mar. 14, 2023, the content of which is incorporated herein by reference in its entirety.

### TECHNICAL FIELD

The present disclosure relates to the technical field of tomographic imaging, and in particular to a digital breast tomosynthesis system, method, apparatus, and storage medium based on X-ray array source.

### BACKGROUND

Breast cancer is the most common cancer among women, and has become the leading cause of cancer-related death among women and the fifth highest cause of cancer-related death worldwide. As the most valuable tool for clinical decision, screening and diagnosis method of breast cancer is essential. At present, digital breast tomosynthesis systems based on X-ray tomosynthesis are commonly used for early-stage breast cancer screening, and pseudo-three-dimensional imaging results are obtained through scanning at limited angles. However, these systems still suffer from several drawbacks. First, X-ray exposes the human body to ionizing radiation, and the radiation dose from the existing digital breast tomosynthesis systems based on X-ray tomosynthesis is not negligible. Second, the existing digital breast tomosynthesis systems use an X-ray source for rotational scanning with mechanical dithering during rotation, which may introduce artifacts and cause reconstruction errors. Third, the scanning range of the existing digital breast tomosynthesis systems is only 11-60°, as a result, projection data are severely incomplete, which will lead to poor quality of reconstructed images, and the inability to accurately reconstruct microcalcifications and other pathological signals, resulting in misdiagnosis and missed diagnosis.

In view of the above defects, some technical solutions have made improvements. For example, a technical solution adopts a single-array ray source to replace the thermal X-ray source, so no rotation is required during image acquisition. However, due to a limited scanning angle, the single-array X-ray source still cannot effectively improve longitudinal resolution of images. Moreover, since plane X-ray array sources are individually packaged, a spacing between the plane X-ray array sources cannot be ignored, which will further cause incomplete projection data and reduce the reconstruction quality.

### SUMMARY

The present disclosure provides a digital breast tomosynthesis system, method, apparatus, and storage medium based on X-ray array source, which can realize virtual rotational projection of an imaging object without moving parts, and can obtain more longitudinal projection information through the plane X-ray array source, thereby improving the longitudinal resolution of reconstructed images.

In order to solve the technical problems, in a first aspect, the present disclosure provides a digital breast tomosynthesis system based on X-ray array source, including a power supply module, an X-ray array source connected to the power supply module, a detection module, a data acquisition module, and an image reconstruction module; where the detection module includes a detection platform for placing an imaging object, a detector located in the detection platform, and a connecting arm configured to connect the plane X-ray array source; the plane X-ray array source is disposed on an opposite side of the detection platform and is configured to emit an X-ray beam to the imaging object; and the plane X-ray array source is a flat-panel X-ray array source or an arc-shaped X-ray array source. When the flat-panel X-ray array source is used, the flat-panel X-ray array source includes at least two ray source units, each ray source unit includes a panel and a plurality of ray sources distributed in an array, the ray sources are arranged on a side of the panel facing the imaging object, and an included angle is formed between adjacent panels, and an angle range of the included angle is 90°-180°. When the arc-shaped X-ray array source is used, only a single X-ray array source is needed, the arc-shaped X-ray array source is a single X-ray array source; the arc-shaped X-ray array source includes a plurality of ray sources distributed in an array, the arc-shaped X-ray array source surrounds the imaging object in a semi-enclosed shape, the ray sources are arranged on a side of the panel facing the imaging object, and a range of arc angle is 90°-180°; and the detector may be a flat-panel detector or an arc-shaped detector, and the arc-shaped detector is configured to receive the X-ray beam emitted from the plane X-ray array source. After receiving an acquisition instruction from the data acquisition module, the detector acquires projection data of the plane X-ray array source and transmits the acquired projection data to the image reconstruction module; and the image reconstruction module reconstructs the projection data to achieve virtual rotation projection of the imaging object, and a differential relationship is introduced on this basis to finally obtain high-quality reconstructed images.

In some exemplary embodiments, the plane X-ray array source is located above, below, or to a side of the imaging object, and the detector is arranged on a side of the imaging object away from the plane X-ray array source.

In some exemplary embodiments, when the plane X-ray array source is the flat-panel X-ray array source, the flat-panel X-ray array source includes two ray source units, the two source units are both arranged to face the imaging object; the two ray source units are located directly above, directly below, or on a same side of the imaging object, the two ray source units are located on a same side of the imaging object, and the two ray source units are symmetric about a longitudinal central axis of the imaging object.

In some exemplary embodiments, when the plane X-ray array source is the flat-panel X-ray array source, the flat-panel X-ray array source includes three ray source units, and the three ray source units are all arranged to face the imaging object; the flat-panel X-ray array source includes a first ray source unit, and a second ray source unit and a third ray source unit located on both sides of the first ray source unit; where the first ray source unit is located directly above, directly below, or on a same side of the imaging object, and the first ray source unit, the second ray source unit, and the third ray source unit are located on a same side of the imaging object; and the second ray source unit and the third ray source unit are symmetric about the longitudinal central axis of the imaging object.

In some exemplary embodiments, the detection module further includes a pressing plate configured to fix the imaging object.

In some exemplary embodiments, the image reconstruction module includes a data correction unit, a data preprocessing unit, and a reconstruction unit connected in sequence, where the data correction unit is configured to perform correction processing of the projection data; the correction processing includes bright-field correction, dark-field correction, zero-field correction, and detector response correction; the data correction unit includes a determination unit and a correction selection unit; the correction selection unit includes a phantom-based correction module and a phantom-free correction module; the determination unit is configured to determine whether a correction phantom is present in the module; the correction selection unit is configured to select the phantom-based correction module to correct the projection data when a correction phantom is present in the module, and the phantom-free correction module is selected to perform correction processing of the projection data when no correction phantom is present in the module; the data preprocessing unit is configured to preprocess the corrected projection data, the preprocessing includes beam shape correction and light intensity correction; the reconstruction unit is configured to design a differential constraint term based on an angle of virtual rotation projection and a differential relationship equation of the detector, to optimize and solve the differential relationship equation based on the differential constraint term, and to reconstruct the preprocessed projection data to obtain an internal structure of the imaging object.

In some exemplary embodiments, the plane X-ray array source further includes a collimator configured to collimate the ray sources on the ray source units; and the collimator is arranged inside the panel, or the collimator is arranged on a side surface of the panel facing the imaging object.

In a second aspect, the present disclosure provides a digital breast tomosynthesis method based on X-ray array source, the method adopts the aforesaid digital breast tomosynthesis system based on X-ray array source to perform CT imaging, including the following steps: setting imaging parameters; placing the imaging object on the detection platform, and setting an angle between panels of adjacent ray source units, or setting an arc angle for the arc-shaped X-ray array source; Under the encoding template, the ray source of the ray source unit is illuminated in an addressable manner; acquiring projection information of all ray beams emitted by the ray sources under the encoding template through the detector to obtain projection data; obtaining virtual rotation projection of plane X-ray array source relative to the imaging object by rearranging panel angles of the ray source units; and designing a differential constraint term based on an angle of virtual rotation projection and a differential relationship equation of the detector, optimizing and solving the differential relationship equation based on the differential constraint term, and reconstructing the preprocessed projection data to obtain an internal structure of the imaging object.

In some exemplary embodiments, the acquiring projection information of all ray beams emitted by the ray sources under the encoding template through the detector to obtain projection data includes: a series of projections acquired under different encoding templates.

In some exemplary embodiments, an acquiring process of the acquired projection information of all ray beams emitted by the ray sources under the encoding template through the detector is expressed as:

$$b = SAf = [ \begin{matrix}0 & 1 & ⋯ & 1 & 1 \\ 1 & 1 & ⋯ & 1 & 0 \\ & & ⋱ & & \\ 0 & 0 & ⋯ & 1 & 1 \\ 1 & 1 & ⋯ & 0 & 0\end{matrix} ] [ \begin{matrix}{P}_{11} , {P}_{12} , … , {P}_{1 N} \\ {P}_{21} , {P}_{22} , … , {P}_{2 N} \\ ⋯ \\ {P}_{M 1} , {P}_{M 2} , … , {P}_{MN}\end{matrix} ] = SP$$

where b denotes measurement data; S denotes a sampling matrix, Sϵ [symbol] ^{K×M}; denotes a number of light sources; l denotes that the lightened source at the corresponding position is illuminated in k-th measurements; P denotes a projection data matrix, that is, projection data from each point source, which is mathematically equal to Af; A denotes a known system matrix, and f denotes an image to be reconstructed; where Pᵢⱼ denotes the measurement data received by a i^{th} detector from a j^{th} lightened source.

In some exemplary embodiments, the obtaining virtual rotation projection of plane X-ray array source relative to the imaging object by rearranging panel angles of the ray source units, including: the cone beams are decoupled using encoded templates to obtain single-point cone beams, and parallel beams at different angles are then obtained by rearranging projections from the ray source units to obtain the virtual rotation projection.

The present disclosure further provides an electronic device, including at least one processor, and a memory in communication connection with the at least one processor; where the memory stores an instruction executable by the at least one processor, and when being executed by the at least one processor, the instruction causes the at least one processor to execute the aforesaid digital breast tomosynthesis method based on X-ray array source.

The present disclosure further provides a computer-readable storage medium storing a computer program, where the computer program, when being executed by a processor, implements the aforesaid digital breast tomosynthesis method based on X-ray array source.

The technical solutions provided by the present disclosure at least have the following advantages.

The embodiments of the present disclosure provide a digital breast tomosynthesis system, method, apparatus, and storage medium based on X-ray array source. The digital breast tomosynthesis system includes a power supply module, an X-ray array source connected to the power supply module, a detection module, a data acquisition module, and an image reconstruction module; where the detection module includes a detection platform for placing an imaging object, a detector located in the detection platform, and a connecting arm configured to connect the plane X-ray array source; the plane X-ray array source is disposed on an opposite side of the detection platform and is configured to emit an X-ray beam to the imaging object; and the plane X-ray array source may be a flat-panel X-ray array source or an arc-shaped X-ray array source. When the flat-panel X-ray array source is used, the flat-panel X-ray array source includes at least two ray source units, each ray source unit includes a panel and a plurality of ray sources distributed in an array, the ray sources are arranged on a side of the panel facing the imaging object, and an included angle is formed between adjacent panels, and an angle range of the included angle is 90°-180°. When the arc-shaped X-ray array source is used, only a single X-ray array source is needed, the plane X-ray array source partially surrounds the imaging object, and a range of arc angle is 90°-180°. The detector is configured to receive the X-ray beam emitted from the plane X-ray array source, and may be a flat-panel detector or an arc-shaped detector. After receiving an acquisition instruction from the data acquisition module, the detector acquires projection data of the plane X-ray array source and transmits the acquired projection data to the image reconstruction module; and the image reconstruction module reconstructs the projection data to achieve virtual rotation projection of the imaging object.

The digital breast tomosynthesis system based on X-ray array source provided by the present disclosure has the following technical benefits: first, by arranging a plurality of sets of flat-panel X-ray array sources or arc-shaped X-ray array sources surrounding the imaging object, a scanning range is greatly expanded; second, by causing each ray source to cover only a portion of the imaging object and using encoding light emission, extremely low-dose scanning can be achieved, thereby reducing a radiation risk; third, decoupling is performed according to measurement data based on coded array beam imaging to obtain a cone-beam projection of a single point source, and parallel beam projections of different angles are generated according to the angles, so as to obtain a virtual rotation projection of the imaging object; and finally, the angle of measurement data and the differential relationship of the detector are introduced by the design of the reconstruction module, such that the resolution and imaging quality of a reconstruction image are improved, and a number of encoding templates is reduced.

### BRIEF DESCRIPTION OF THE DRAWINGS

One or more embodiments are exemplarily described by drawings in corresponding accompanying drawings, and such exemplary description does not constitute a limitation on the embodiments. The figures in the accompanying drawings do not constitute a scale limitation unless otherwise stated.

FIG. 1 is a block diagram of a structure of a digital breast tomosynthesis system based on X-ray array source according to one embodiment of the present disclosure.

FIG. 2 is a schematic structural diagram of a digital breast tomosynthesis system based on X-ray array source according to one embodiment of the present disclosure.

FIG. 3 is a schematic structural diagram of a digital breast tomosynthesis system based on X-ray array source according to another embodiment of the present disclosure.

FIG. 4a is a schematic diagram illustrating the placement of a digital breast tomosynthesis system based on X-ray array source for digital breast tomosynthesis according to one embodiment of the present disclosure.

FIG. 4b is a schematic diagram illustrating the placement of a digital breast tomosynthesis system based on X-ray array source for digital breast tomosynthesis according to another embodiment of the present disclosure.

FIG. 4c is a schematic diagram illustrating the placement of a digital breast tomosynthesis system based on X-ray array source for digital breast tomosynthesis according to yet another embodiment of the present disclosure.

FIG. 5a is a schematic structural diagram of a digital breast tomosynthesis system based on X-ray array source according to yet another embodiment of the present disclosure.

FIG. 5b is a schematic structural diagram of a digital breast tomosynthesis system based on X-ray array source according to yet another embodiment of the present disclosure.

FIG. 6 is a schematic structural diagram of a digital breast tomosynthesis system based on X-ray array source according to yet another embodiment of the present disclosure.

FIG. 7 is a schematic structural diagram of a digital breast tomosynthesis system based on X-ray array source according to yet another embodiment of the present disclosure.

FIG. 8 is a cross-sectional view of a data acquisition process according to one embodiment of the present disclosure.

FIG. 9 is a cross-sectional view of a data acquisition process according to another embodiment of the present disclosure.

FIG. 10 is a cross-sectional view of a data acquisition process according to yet another embodiment of the present disclosure.

FIG. 11 is a schematic diagram of a correction process of a data correction unit according to one embodiment of the present disclosure.

FIG. 12 is a flowchart of a digital breast tomosynthesis method based on X-ray array source according to one embodiment of the present disclosure.

FIG. 13 is a flowchart of a digital breast tomosynthesis data acquisition and reconstruction method according to one embodiment of the present disclosure.

FIG. 14 is a schematic diagram of encoded sampling of a digital breast tomosynthesis system based on X-ray array source according to one embodiment of the present disclosure.

FIG. 15 is a schematic structural diagram of an electronic device according to one embodiment of the present disclosure.

### DETAILED DESCRIPTIONS OF THE EMBODIMENTS

As can be seen from the background art, the existing digital breast tomosynthesis systems with static structure only adopt a single-array ray source to replace the thermal-source ray source, without the need for rotation during an acquisition process. However, the single-array ray source is still unable to improve longitudinal resolution, and the spacing between individually packaged plane X-ray array sources will result in incomplete projection data and cause image degradation.

Currently, there are technical solutions that use a plane X-ray array source to replace the thermal-source ray source, the plane X-ray array source is arranged opposite a detector to receive measurement signals, and the received signals are transmitted to a reconstruction system via detector acquisition software. Compared with the single-array ray source, using a plane X-ray array source to replace the thermal-source ray source can improve the longitudinal resolution to a certain extent. However, a scanning range is still limited by simply arranging a flat-panel X-ray array source and a detector in parallel, and the reconstruction quality of fine structures cannot be guaranteed.

In order to solve the technical problems, the embodiments of the present disclosure provide a digital breast tomosynthesis system, method, apparatus, and storage medium based on X-ray array source. The digital breast tomosynthesis system includes a power supply module, an X-ray array source connected to the power supply module, a detection module, a data acquisition module, and an image reconstruction module; where the detection module includes a detection platform for placing an imaging object, a detector located in the detection platform, and a connecting arm configured to connect the plane X-ray array source; the plane X-ray array source is disposed on an opposite side of the detection platform and is configured to emit an X-ray beam to the imaging object; and the plane X-ray array source may be a flat-panel X-ray array source or an arc-shaped X-ray array source. When the flat-panel X-ray array source is used, the flat-panel X-ray array source includes at least two ray source units, each ray source unit includes a panel and a plurality of ray sources distributed in an array, the ray sources are arranged on a side of the panel facing the imaging object, and an included angle is formed between adjacent panels, and an angle range of the included angle is 90°-180°. When the arc-shaped X-ray array source is used, only a single X-ray array source is needed, the plane X-ray array source partially surrounds the imaging object, and a range of arc angle is 90°-180°; the detector may be a flat-panel detector or an arc-shaped detector, and the arc-shaped detector is configured to receive the X-ray beam emitted from the plane X-ray array source. After receiving an acquisition instruction from the data acquisition module, the detector acquires projection data of the plane X-ray array source and transmits the acquired projection data to the image reconstruction module; and the image reconstruction module reconstructs the projection data to achieve virtual rotation projection of the imaging object, and a differential relationship is introduced on this basis to finally obtain high-quality reconstructed images.

The embodiments of the present disclosure provide a digital breast tomosynthesis system, method, apparatus, and storage medium based on X-ray array source, thereby obtaining more longitudinal projection information and improving the longitudinal resolution. First, by arranging a plurality of sets of flat-panel X-ray array sources or arc-shaped X-ray array sources surrounding the imaging object, a scanning range is greatly expanded; second, with each ray source to cover only a portion of the imaging object and using encoding templates, extremely low-dose scanning can be achieved, thereby reducing a radiation risk; third, decoupling is performed according to measurement data based on coded array beam imaging to obtain a cone-beam projection of a single point source, and parallel beam projections of different angles are generated according to the angles, so as to obtain a virtual rotation projection of the imaging object; and finally, the angle of measurement data and the differential relationship of the detector are introduced by the design of the reconstruction system, such that the reconstruction quality is improved.

The embodiments of the present disclosure will be described in detail below with reference to accompanying drawings. However, those of ordinary skill in the art may understand that in each embodiment of the present disclosure, many technical details have been put forward in order to make readers better understand the present disclosure. Nevertheless, even without these technical details and various changes and modifications based on the embodiments below, technical solutions to be protected required by the present disclosure may be basically implemented.

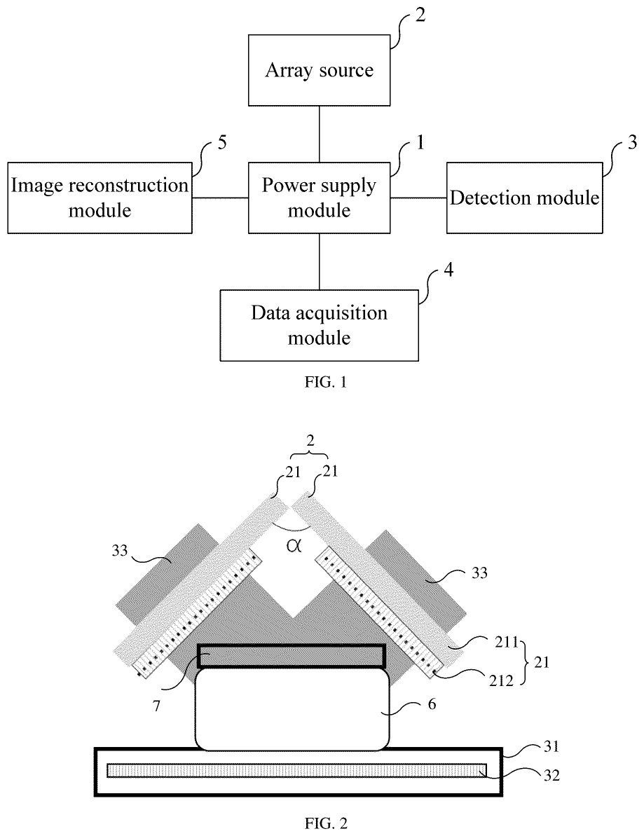

As shown in FIGS. 1 and 2, this embodiment provides a digital breast tomosynthesis system based on X-ray array source, including: a power supply module 1, a plane X-ray array source 2 connected to the power supply module 1, a detection module 3, a data acquisition module 4, and an image reconstruction module 5; where the detection module 3 includes a detection platform 31 configured to hold an imaging object 6, a detector 32 located in the detection platform 31, and a connecting arm 33 configured to connect to the plane X-ray array source 2; and the plane X-ray array source 2 is located on an opposite side of the detection platform 31 and configured to emit an X-ray beam toward the imaging object 6.

As shown in FIG. 1, the power supply module 1 is connected to the plane X-ray array source 2, the detection module 3, the data acquisition module 4, and the image reconstruction module 5, respectively, and the power supply module 1 is mainly configured to provide high voltage and ordinary power supply required by each module. A structural diagram of the digital breast tomosynthesis system based on X-ray array source according to this embodiment is shown in FIG. 2.

It should be noted that the plane X-ray array source 2 may be implemented as a flat-panel X-ray array source or an arc-shaped X-ray array source. FIG. 2 illustrates a situation where the plane X-ray array source in the digital breast tomosynthesis system is a flat-panel X-ray array source.

When the plane X-ray array source 2 is a flat-panel X-ray array source, as shown in FIG. 2, the plane X-ray array source 2 includes at least two ray source units 21; each ray source unit 21 includes a panel 211 and a plurality of ray sources 212 distributed in an array, the ray sources 212 are arranged on the side the panel 211 facing the imaging object 6, an included angle is formed between adjacent panels 211, and a range of the included angle is 90°-180°; and the detector 32 is configured to receive the X-ray beam emitted from the flat-panel X-ray array source, the detector 32 acquires projection data of the flat-panel X-ray array source and transmits the acquired projection data to the image reconstruction module 5 after receiving an acquisition instruction from the data acquisition module 4, and the image reconstruction module 5 is configured to reconstruct the projection data to achieve virtual rotation projection of the imaging object 6, and a differential relationship is introduced on this basis to finally obtain high-quality reconstructed images.

As shown in FIG. 2, the imaging object 6 is placed on the inspection platform 31, the detector 32 is arranged inside the inspection platform 31, the plane X-ray array source 2 (the flat-panel X-ray array source) and the inspection platform 31 are connected by the connecting arm 33, that is, the flat-panel X-ray array source is fixed above the inspection platform 31 via the connecting arm 33; and specifically, the connecting arm 33 may be a telescopic connecting rod, and a position of the flat-panel X-ray array source can be adjusted by adjusting length and angle of the telescopic connecting rod. The inspection platform 31 may be a support table or a support plate, the detector 32 is embedded in the support table or the support plate, and the detector 32 may be a flat-panel detector or an arc-shaped detector.

As shown in FIG. 2 again, the plane X-ray array source 2 is located above the inspection platform 31 and configured to emit an X-ray beam toward the imaging object 6; the plane X-ray array source 2 includes at least two ray source units 21, and FIG. 2 illustrates an example in which the plane X-ray array source 2 includes two ray source units 21, where the two ray source units 21 are arranged at a certain angle, and the two ray source units 21 are both oriented toward the imaging object 6. The ray sources 212 are distributed in a matrix on the panel 211 of each ray source unit 21, and an included angle is formed between adjacent panels 211, that is, there is an included angle between the two ray source units 21. As shown in FIG. 2, the two ray source units 21 are arranged above the imaging object 6 at an included angle α, and a range of the included angle α is 90°-180°. For example, the included angle a may be 90°, 120°, 150°, or 180°. Since the two ray source units 21 are arranged at a certain angle, the X-ray beam emitted from the ray sources 212 cover the imaging object 6, and more longitudinal projection information can be obtained, thereby improving the longitudinal resolution.

It should be noted that the included angle a may be adjusted according to different imaging requirements.

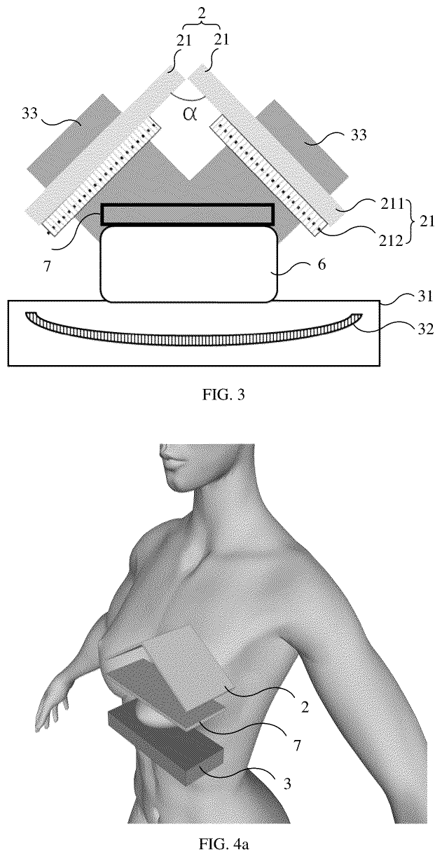

It should also be noted that, during the data acquisition process, the detector 32 receives information of all X-ray beams emitted from the plane X-ray array source 2 under an encoding template. The detector 32 may be a flat-panel detector or an arc-shaped detector. When the arc-shaped detector is used, it needs to be arranged inside the inspection platform 31, as shown in FIG. 3. In some embodiments, the plane X-ray array source 2 is located above, below, or to a side of the imaging object 6, and the detector 32 is arranged on the side of the imaging object 6 away from the plane X-ray array source 2. That is, in some embodiments, the plane X-ray array source 2 may be located above the imaging object 6, and the detector 32 may be located below the imaging object 6.

In other embodiments, the plane X-ray array source 2 may be located below the imaging object 6, and the detector 32 may be located above the imaging object 6.

In yet other embodiments, the plane X-ray array source 2 may be located on the side of the imaging object 6, and the detector 32 may be located on the side of the imaging object 6 away from the plane X-ray array source 2.

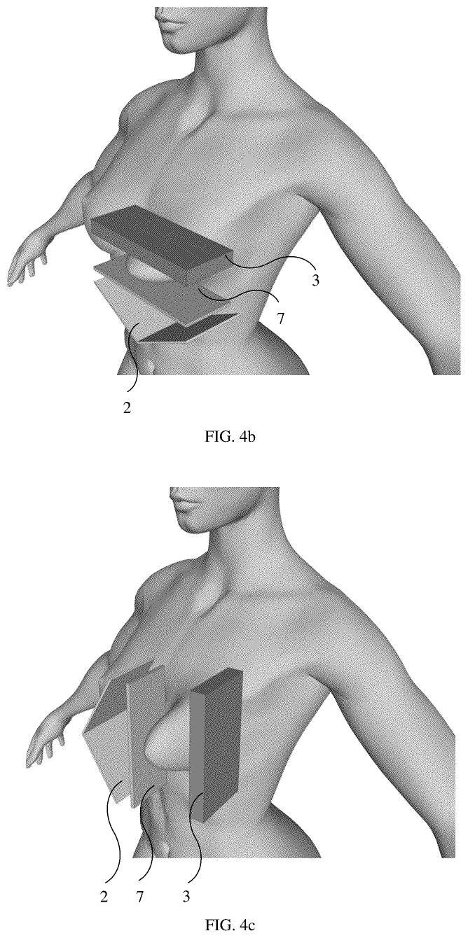

As shown in FIGS. 4a-4c, FIGS. 4a-4c are schematic diagrams illustrating various placement modes of the digital breast tomosynthesis system based on X-ray array source in practical applications provided by the present disclosure. In FIGS. 4a-4c, the plane X-ray array source 2 is a flat-panel X-ray array source, and the plane X-ray array source 2 includes two flat-panel X-ray array sources for illustrative purposes.

When the plane X-ray array source 2 is the flat-panel X-ray array source, the plane X-ray array source 2 includes two ray source units 21, the two source units 21 are both arranged to face the imaging object 6; the two ray source units 21 are located directly above, directly below, or on the side of the imaging object 6, the two ray source units 21 are located on a same side of the imaging object 6, and the two ray source units 21 are symmetric about a longitudinal central axis of the imaging object 6. As shown in FIG. 4a, the two ray source units 21 are located directly above the imaging object 6, and the detector 32 (the detection module 3 is shown in FIG. 4a, and the detector 32 is arranged inside the detection module 3) is located directly below the imaging object 6; as shown in FIG. 4b, two ray source units 21 are located directly below the imaging object 6, the detector 32 (the detection module 3 is shown in FIG. 4b, and the detector 32 is arranged inside the detection module 3) is located directly above the imaging object 6; as shown in FIG. 4c, the two ray source units 21 are located on the side of the imaging object 6, the two ray source units 21 are both located on the same side of the imaging object 6, the detector 32 (the detection module 3 is shown in FIG. 4c, and the detector 32 is arranged inside the detection module 3) is located on the opposite side of the imaging object 6, the two ray source units 21 are arranged opposite to the detector 32, and the imaging object 6 is located between the ray source units 21 and the detector 32.

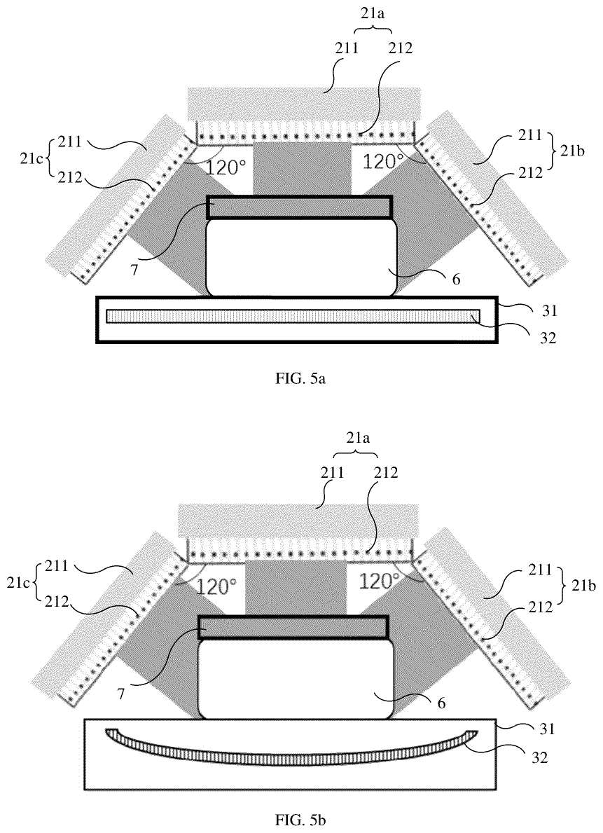

Of course, it should be understood that the plane X-ray array source 2 may also include three ray source units 21. In some embodiments, the plane X-ray array source 2 includes three ray source units 21, and the three ray source units 21 are all arranged to face the imaging object 6; as shown in FIG. 5a, the plane X-ray array source includes a first ray source unit 21a, and a second ray source unit 21b and a third ray source unit 21c respectively located on both sides of the first ray source unit 21a; where the first ray source unit 21a is located directly above the imaging object 6, and the second ray source unit 21b and the third ray source unit 21c are symmetric about the longitudinal central axis of the imaging object 6. As shown in FIG. 5a, an angle between the first ray source unit 21a and the second ray source unit 21b is 60°, and an angle between the first ray source unit 21a and the third ray source unit 21c is also 60°. That is, an included angle between each pair of the three ray source units 21 is 120°. In this way, the X-ray beams emitted by the ray sources 212 of the three ray source units 21 cover the imaging object 6, and more longitudinal projection information can be obtained, thereby further improving the longitudinal resolution.

It should be understood that when the plane X-ray array source 2 includes three ray source units 21, the detector 32 may also be an arc-shaped detector embedded in the detection platform 31, as shown in FIG. 5b.

In some embodiments, the plane X-ray array source 2 further includes a collimator configured to collimate the ray sources 212 on the ray source units 21. The collimator may be arranged inside the panel 211, or on a surface of the panel 211 facing the imaging object 6, that is, the collimator may be placed in front of the plane X-ray array source 2 or a built-in collimator may be arranged in the plane X-ray array source to meet the imaging requirements.

As described earlier, the plane X-ray array source 2 may be a flat-panel X-ray array source or an arc-shaped X-ray array source.

When the plane X-ray array source 2 is an arc-shaped X-ray array source, the arc-shaped X-ray array source is a single X-ray array source; the arc-shaped X-ray array source includes a plurality of ray sources 212 distributed in an array, the arc-shaped X-ray array source surrounds the imaging object 6 in a semi-enclosed shape, and the ray sources 212 are arranged on the side of the panel 211 facing the imaging object 6; and an arc angle range of the arc-shaped X-ray array source is 90°-180°.

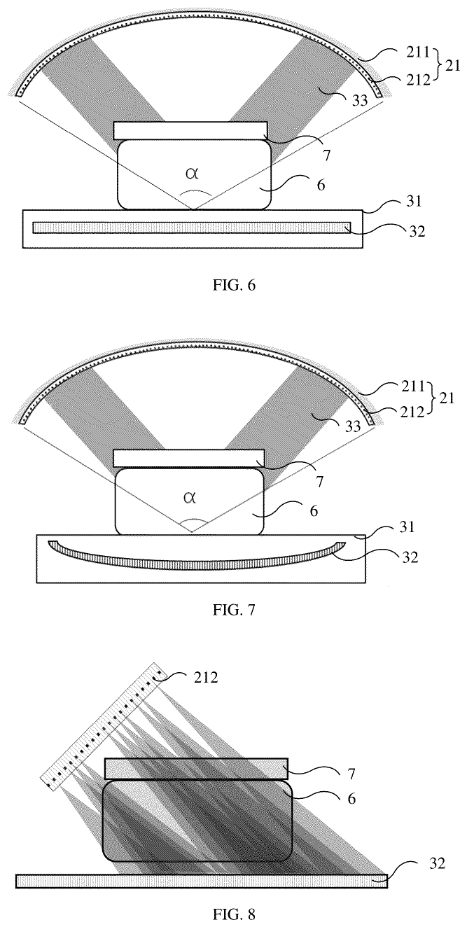

As shown in FIGS. 6 and 7, an arc-shaped panel of the arc-shaped X-ray array source may be an integrated structure, where the panel 211 (arc-shaped panel) is arranged above the detection platform 31 and the imaging object 6, the ray source units 21 are fixed on the side of the panel 211 facing the imaging object 6, and an included angle is formed between adjacent ray source units 21. As shown in FIG. 6, two end portions of the panel 211 form an angle α with a bottom center of the imaging object 6, where a range of the angle α is 90°-180°.

As shown in FIG. 6, the ray sources 212 of the ray source units 21 are arranged in a matrix on the side of the panel 211 (arc-shaped panel) facing the imaging object 6, each ray source 212 emits a cone beam that covers a portion of the imaging object 6, and when all ray sources 212 (light sources) are illuminated, the entire imaging object 6 can be completely covered. The plane X-ray array source 2 may be controlled via the power supply module 1 to emit X-ray beams according to a specific coded pattern, such that the plane X-ray array source 2 emits the X-ray beams under different encoding templates. FIG. 7 is a schematic structural diagram of the digital breast tomosynthesis system based on X-ray array source according to this embodiment when an arc-shaped panel and an arc-shaped detector are used.

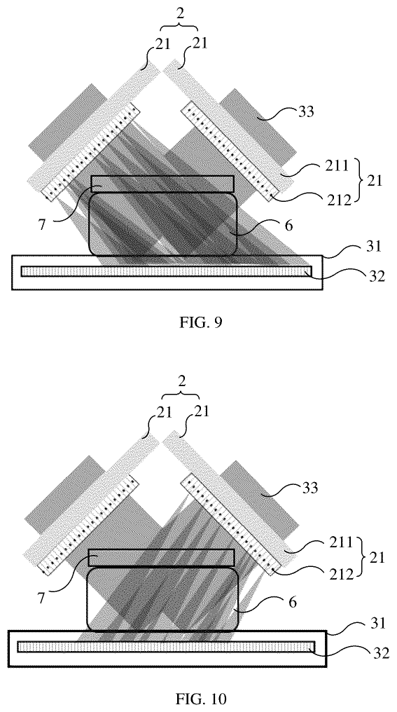

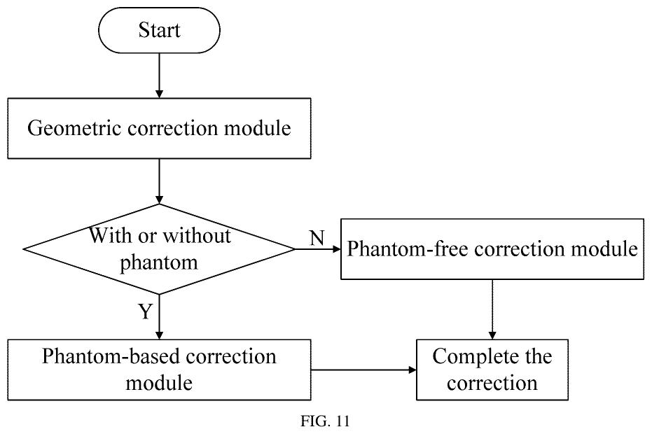

In some embodiments, the detection module 3 further includes a pressing plate 7 configured to fix the imaging object 6. The pressing plate 7 is pressed against a top of the imaging object 6 to fix the imaging object 6. The ray sources 212 of the ray source units 21 are capable of emitting the X-ray beams through the pressing plate 7 toward the imaging object 6. As shown in FIGS. 8-10, in the data acquisition system, the imaging object 6 is placed on the detection platform 31, and the imaging object 6 is fixed with the pressing plate 7 from above. Under a current encoding state of the plane X-ray array source 2, the ray sources 212 of the ray source units 21 emit ray beams (cone beams) to cover a portion of the imaging object 6, and the detector 32 receives all beams emitted by the ray sources 212 of the plane X-ray array source 2. During the acquisition process, one ray source unit 21 is illuminated first. After data acquisition under all encoding modes is completed, another ray source unit 21 is illuminated to complete all the preset data acquisition.

During the data acquisition process, each ray source unit 21 emits a cone beam that covers a portion of the imaging object 6, and the entire imaging object 6 can be completely covered when all the light sources are illuminated. A specific encoding template is controlled via the power supply module 1, such that the ray source unit 21 emits the beams under different encoding templates. During the acquisition process, the detector 32 receives information of all ray beams under the encoding templates.

It should be noted that, during the data acquisition process, the illumination mode can be customized according to the imaging object 6, or a commonly used compressed pattern, such as Hadamard pattern, orthogonal pattern, or Gaussian random matrix pattern, may be used.

After acquisition is completed, the cone beams are decoupled using encoding illumination to obtain single-point cone beams, and parallel beams at different angles are then obtained by rearranging projections to obtain the virtual rotation projection.

In some embodiments, the image reconstruction module 5 includes a data correction unit, a data preprocessing unit, and a reconstruction unit connected in sequence, where the data correction unit is configured to perform correction processing of the projection data; the correction processing includes bright-field correction, dark-field correction, zero-field correction, and detector response correction; the data correction unit includes a determination unit and a correction selection unit; the correction selection unit includes a phantom-based correction module and a phantom-free correction module; the determination unit is configured to determine whether a correction phantom is present in the module; the correction selection unit is configured to select the phantom-based correction module to correct the projection data when a correction phantom is present in the module, and the phantom-free correction module is selected to perform correction processing of the projection data when no correction phantom is present in the module; the data preprocessing unit is configured to preprocess the corrected projection data, the preprocessing includes beam shape correction and light intensity correction; the reconstruction unit is configured to design a differential constraint term based on an angle of virtual rotation projection and a differential relationship equation of the detector, to optimize and solve the differential relationship equation based on the differential constraint term, and to reconstruct the preprocessed projection data to obtain an internal structure of the imaging object.

As shown in FIG. 11, specifically, the image reconstruction module 5 includes a phantom-free correction scheme and a phantom-based correction scheme. The determination unit in the data correction unit first determines whether a correction phantom is present in the module. When a correction phantom is present, the correction selection unit selects a correction method with the correction phantom to perform correction processing of the projection data. When no correction phantom is present, the correction selection unit selects a correction method without the correction phantom to perform the correction processing of the projection data. The data preprocessing unit is configured to deal with the problems caused by inconsistent current levels under different illumination modes.

In summary, the digital breast tomosynthesis system based on X-ray array source provided by the present disclosure has the following technical benefits: first, by arranging a plurality of sets of flat-panel X-ray array sources or arc-shaped X-ray array sources surrounding the imaging object, a scanning range is greatly expanded; second, by causing each ray source to cover only a portion of the imaging object and using encoding light emission, extremely low-dose scanning can be achieved, thereby reducing a radiation risk; third, decoupling is performed according to measurement data based on coded array beam imaging to obtain a cone-beam projection of a single point source, and parallel beam projections of different angles are generated according to the angles, so as to obtain a virtual rotation projection of the imaging object; and finally, the angle of measurement data and the differential relationship of the detector are introduced by the design of the reconstruction module, such that the reconstruction quality is improved.

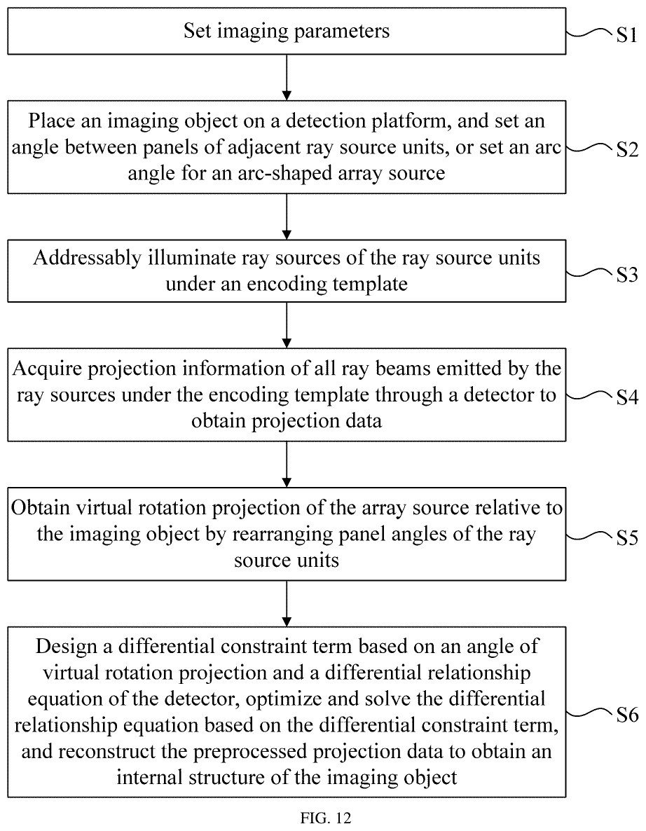

As shown in FIG. 12, an embodiment of the present disclosure also provides a digital breast tomosynthesis method based on X-ray array source, and the digital breast tomosynthesis system based on X-ray array source in the previous embodiment is used to perform CT imaging, including the following steps:
step S1: setting imaging parameters;
step S2: placing the imaging object on the detection platform, and setting an angle between panels of adjacent ray source units, or setting an arc angle for arc-shaped X-ray array source;
step S3: addressably illuminating ray sources of the ray source units under the encoding template;
step S4: acquiring projection information of all ray beams emitted by the ray sources under the encoding template through the detector to obtain projection data;
step S5: obtaining virtual rotation projection of plane X-ray array source relative to the imaging object by rearranging panel angles of the ray source units; and
step S6: designing a differential constraint term based on an angle of virtual rotation projection and a differential relationship equation of the detector, optimizing and solving the differential relationship equation based on the differential constraint term, and reconstructing the preprocessed projection data to obtain an internal structure of the imaging object.

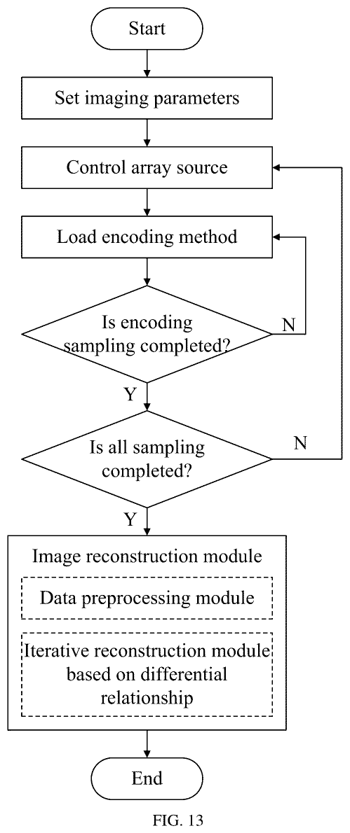

As shown in FIG. 13, during the acquisition process, the imaging parameters are first set, the plane X-ray array source is controlled according to a preset sequence, and different encoding templates are loaded; it is then determined whether data acquisition under the current encoded sampling is completed, data of all plane X-ray array sources are collected, the collected data are inputted into the image reconstruction module, which includes a data preprocessing module (the data preprocessing unit) and an iterative reconstruction module based on the differential relationship (the reconstruction unit).

In some embodiments, the detector acquires the projection information of all ray beams emitted by the ray sources under the encoding template through the detector to obtain projection data, including: the detector samples projection information of all ray beams emitted by the ray sources under different encoding templates to obtain projection data.

Specifically, during the projection generation process, the ray sources are addressably illuminated using a control circuit, where Pattern={S₁,S₂ . . . ,Sᵢ . . . ,S_{N}} denotes a sequence of N designed illumination schemes, and an ideal is expressed as:

$$b = SAf = [ \begin{matrix}0 & 1 & ⋯ & 1 & 1 \\ 1 & 1 & ⋯ & 1 & 0 \\ & & ⋱ & & \\ 0 & 0 & ⋯ & 1 & 1 \\ 1 & 1 & ⋯ & 0 & 0\end{matrix} ] [ \begin{matrix}{P}_{11} , {P}_{12} , … , {P}_{1 N} \\ {P}_{21} , {P}_{22} , … , {P}_{2 N} \\ ⋯ \\ {P}_{M 1} , {P}_{M 2} , … , {P}_{MN}\end{matrix} ] = SP$$

where b denotes measurement data; S denotes a sampling matrix, Sϵ [symbol] ^{K×M}; M denotes a number of light sources; l denotes that the light source at the corresponding position is illuminated in k-th measurements; P denotes a projection data matrix, that is, projection data from each point source, which is mathematically equal to Af; A denotes a known system matrix, and f denotes an image to be reconstructed; where Pᵢⱼ denotes the measurement data received by a i^{th} detector from a j^{th} light source.

A process of recovering the imaging object f from the measurement data b is a typical inverse problem solution. Based on Bayesian estimation, the following optimization equation can be constructed by maximizing a posterior probability:

$$\{ f , P \} = arg {min}_{f , P} ( \frac{w}{2} {‖ SP - b ‖}_{2}^{2} + {α}_{i} {R}_{i} ( f ) + {β}_{j} {R}_{j} ( P ) + {γ}_{k} {R}_{k} ( f , P ) )$$

where w denotes a weight, Rᵢ(f) denotes a regularization term based on an image domain, Rⱼ(P) denotes a regularization term based on a projection domain, and Rₖ(P) denotes a regularization term based on a dual-domain of projection and image.

For the regularization term of image domain, a total variation (TV), a dictionary learning (DL) and other regularization terms based on compressed sensing may be selected based on prior information of the image. For the regularization term of projection domain, the total variation, the TV may be selected as the regularization term.

When designing Rₖ(P), a differential constraint term is designed by leveraging a differential relationship between the projection angle and the detector, for example, the following relationship expression may be used:

$${‖ \frac{\partial BP}{\partial θ} - \frac{\partial BA ( ( x \cdot {θ}^{⊥} ) f ) )}{\partial t} ‖}_{2}^{2}$$

where in a two-dimensional case, θ^{⊥}=(sin θ, −cos θ) and x are coordinates of an image, B(⋅) denotes a function that converts aliased projection data into virtual rotation projection, and t denotes a detector index.

The above optimization problem can be solved using an alternating direction method of multipliers.

The problem can also be taken as an optimization problem based on partial differential equation constraints and can be expressed as:

$$\{ \begin{matrix}{min}_{( y ( x ) , u ( x ) ) ∈ Y \times U} J ( y ( x ) , u ( x ) ) \\ subject to \{ \begin{matrix}F ( y ( x ) , u ( x ) ) = 0 , in Ω \\ u ( x ) ∈ {U}_{ad}\end{matrix}\end{matrix}$$

where y(x) denotes a state function, u(x) denotes a control function, J denotes an objective function in integral form, and F denotes a partial differential equation function. The quality of image reconstruction can be constrained by applying the known differential relationship between the projection angle and the detector as the control function u(x).

In this framework, the above problem can be further expressed as:

$$\{ \begin{matrix}{min}_{f , P} ( \frac{w}{2} {‖ SP - b ‖}_{2}^{2} + {α}_{i} {R}_{i} ( f ) + {β}_{j} {R}_{j} ( P ) + {γ}_{k} {‖ u ( P ) ‖}_{L 2 ( Ω )}^{p} ) ) \\ subject to \{ \begin{matrix}F ( y ( x ) , u ( x ) ) = 0 , in Ω \\ u ( x ) ∈ {U}_{ad}\end{matrix}\end{matrix}$$

where F denotes a partial differential equation; and by introducing a virtual detector plane and a virtual rotation center, the above problem can be specifically expressed as:

$$\{ \begin{matrix}{min}_{f P} ( \frac{w}{2} {‖ SP - b ‖}_{2}^{2} + {α}_{i} {R}_{i} ( f ) + {β}_{j} {R}_{j} ( P ) + {γ}_{k} {‖ u ( P ) ‖}_{L 2 ( Ω )}^{p} ) ) \\ subject to \{ \begin{matrix}\frac{{\partial }^{2} P}{\partial {o}^{2}} + \frac{2 {cos}^{2} θ}{( H - d )} \frac{{\partial }^{2} P}{\partial θ \partial 0} + \frac{( H + d ) {cos}^{4} θ}{{( H - d )}^{3}} \frac{{\partial }^{2} P}{\partial {θ}^{2}} - {( \frac{H}{H - d} )}^{2} \frac{{\partial }^{2} P}{\partial {η}^{2}} = u ( P ) , in Ω \\ u ( P ) ∈ {U}_{ad}\end{matrix}\end{matrix}$$

where P denotes projection measurement data, η denotes a detector index, H denotes a height of a light source from the detector, d denotes a height of a virtual rotation center from the detector, o denotes a virtual detector plane index, θ denotes an angle between a ray and a detector normal, and

$${‖ u ( P ) ‖}_{L 2 ( Ω )}^{p}$$

a p-norm or u(P).

In addition to the above,

$$u ( P ) = \frac{\partial BP}{\partial θ} - \frac{\partial BA ( ( x \cdot {θ}^{⊥} ) f ) )}{\partial t} or u ( P ) = \frac{\partial P}{\partial o} + \frac{{cos}^{2} θ}{( H - d )} ( \frac{\partial P}{\partial θ} - \frac{H}{{cos}^{2} θ} \frac{\partial P}{\partial η} )$$

may also be selected to construct or optimize the equation for solution.

For solving the above optimization problem, traditional iterative methods may be adopted, or deep learning techniques may be embedded, a physics-informed neural network (PINN) may be used to the partial differential equation, and the solution is brought into an iterative framework for solution.

In some embodiments, the obtaining virtual rotation projection of plane X-ray array source relative to the imaging object by rearranging panel angles of the ray source units, including: the cone beams are decoupled using encoding templates to obtain single-point cone beams, and parallel beams at different angles are then obtained by rearranging projections from the ray source units to obtain the virtual rotation projection, and a differential relationship is introduced on this basis to finally obtain high-quality reconstructed images.

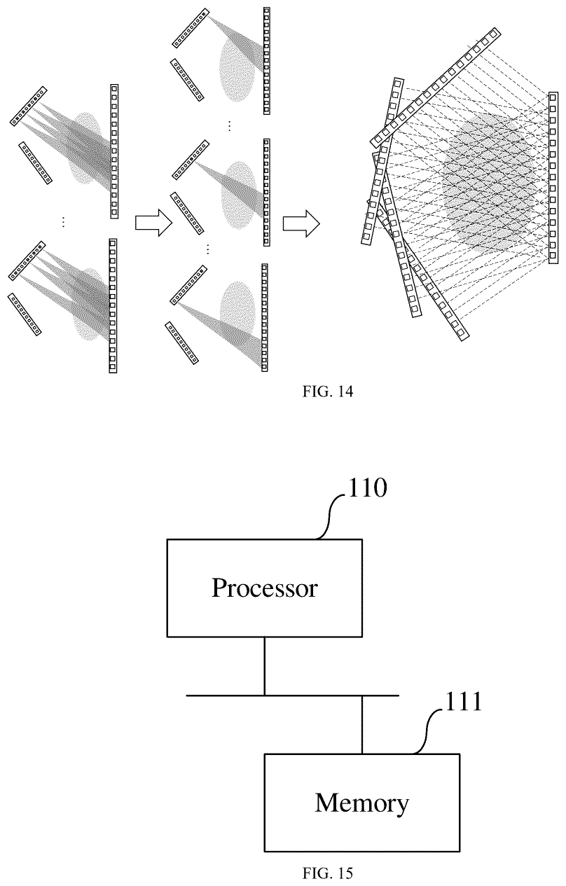

FIG. 14 shows an example of encoded sampling of a digital breast tomosynthesis method based on X-ray array source. First, the encoded sampling process is performed: a ray source unit emits cone beams to cover a portion of the imaging object, and the entire imaging object is fully covered when all light sources are illuminated; a specific coded pattern is controlled via the power supply module, such that the plane X-ray array source emits the beams under different encoding templates, and the detector receives information from the plane X-ray array source under the encoding templates. After the sampling under all the encoding templates is completed, single-point cone beam projection can be obtained by de-aliasing, and virtual rotation projection of plane X-ray array source relative to the imaging object is obtained by rearranging the angle.

The digital breast tomosynthesis method based on X-ray array source provided in the embodiment uses the digital breast tomosynthesis system based on X-ray array source in the above embodiment to perform CT imaging, specifically, de-coupling is performed according to measurement data based on coded array beam imaging to obtain a cone-beam projection of a single point source, and parallel beam projections of different angles are generated according to the angles, so as to obtain a virtual rotation projection of the imaging object; and finally, the angle of measurement data and the differential relationship of the detector are introduced by the design of the reconstruction system, such that the reconstruction quality is improved.

As shown in FIG. 15, another embodiment of the present disclosure provides an electronic device, including at least one processor 110, and a memory 111 in communication connection with the at least one processor; where the memory 111 stores an instruction executable by the at least one processor 110, and when being executed by the at least one processor 110, the instruction causes the at least one processor 110 to execute any of the above method embodiments.

The memory 111 and the processor 110 are connected through a bus, and the bus may include interconnected buses and bridges unlimited in the number, and the bus connects various circuits of one or more processors 110 and the memory 111 together. The bus may further connect various other circuits such as peripheral devices, voltage regulators, and power management circuits, which are well known in the art, and therefore are not further described herein. A bus interface provides an interface between the bus and a transceiver. The transceiver may be one or more components, such as a plurality of receivers and senders, providing a unit for being in communication with various other apparatuses on a transmission medium. Data processed by the processor 110 are transmitted on a wireless medium through an antenna. Further, the antenna also receives the data and transmits the data to the processor 110.

The processor 110 is responsible for managing the bus and general processing, and may also provide various functions, including timing, peripheral interfaces, voltage regulation, power management and other control functions. The memory 111 may be used for storing data used by the processor 110 during operations.

Based on the above technical solutions, the embodiments of the present disclosure provide a digital breast tomosynthesis system, method, apparatus, and storage medium based on X-ray array source. The digital breast tomosynthesis system includes a power supply module, an X-ray array source connected to the power supply module, a detection module, a data acquisition module, and an image reconstruction module; where the detection module includes a detection platform for placing an imaging object, a detector located in the detection platform, and a connecting arm configured to connect the plane X-ray array source; the plane X-ray array source is disposed on an opposite side of the detection platform and is configured to emit an X-ray beam to the imaging object; and the plane X-ray array source may be a flat-panel X-ray array source or an arc-shaped X-ray array source. When the flat-panel X-ray array source is used, the flat-panel X-ray array source includes at least two ray source units, each ray source unit includes a panel and a plurality of ray sources arranged in an array, the ray sources are arranged on a side of the panel facing the imaging object, and an included angle is formed between adjacent panels, and an angle range of the included angle is 90°-180°. When the arc-shaped X-ray array source is used, only a single X-ray array source is needed, the plane X-ray array source partially surrounds the imaging object, and a range of arc angle is 90°-180°; the detector is configured to receive the X-ray beam emitted from the plane X-ray array source, and may be a flat-panel detector or an arc-shaped detector. After receiving an acquisition instruction from the data acquisition module, the detector acquires projection data of the plane X-ray array source and transmits the acquired projection data to the image reconstruction module; and the image reconstruction module reconstructs the projection data to achieve virtual rotation projection of the imaging object, and a differential relationship is introduced on this basis to finally obtain high-quality reconstructed images.

The digital breast tomosynthesis system based on X-ray array source provided by the present disclosure has the following technical benefits: first, by arranging a plurality of sets of flat-panel plane X-ray array sources or arc-shaped X-ray array sources surrounding the imaging object, a scanning range is greatly expanded; second, with each ray source to cover only a portion of the imaging object and using encoding light emission, extremely low-dose scanning can be achieved, thereby reducing a radiation risk; third, decoupling is performed according to measurement data based on coded array beam imaging to obtain a cone-beam projection of a single point source, and parallel beam projections of different angles are generated according to the angles, so as to obtain a virtual rotation projection of the imaging object; and finally, the angle of measurement data and the differential relationship of the detector are introduced by the design of the reconstruction module, such that the reconstruction quality is improved.

It may be understood by those skilled in the art that the above embodiments are particular embodiments to implement the present disclosure, and in a practical application, various changes can be made to the form and details without departing from the spirit and scope of the present disclosure. It should be understood by those skilled in the art that the above-mentioned embodiments are specific examples for implementing the present disclosure. In practical applications, various changes may be made in form and detail without departing from the spirit and scope of the present disclosure. Those skilled in the art may make variations or modifications without departing from the spirit and scope of the present disclosure, therefore, the scope of protection of the present disclosure shall be subject to the scope of protection as defined by the claims.

What is claimed is:

1. A digital breast tomosynthesis system based on an X-ray array source, comprising:
a power supply module, an X-ray array source connected to the power supply module, a detection module, a data acquisition module, and an image reconstruction module; wherein
the detection module comprises a detection platform for placing an imaging object, a detector located in the detection platform, and a connecting arm configured to connect the X-ray array source;
the X-ray array source is disposed on an opposite side of the detection platform and is configured to emit an X-ray beam to the imaging object; and a plane X-ray array source is a flat-panel plane X-ray array source or an arc-shaped X-ray array source;
when the flat-panel plane X-ray array source is used, the flat-panel plane X-ray array source comprises at least two ray source units; wherein each ray source unit comprises a panel and a plurality of ray sources distributed in an array, the ray sources are arranged on a side of the panel facing the imaging object, and an included angle is formed between adjacent panels, and an angle range of the included angle is 90°-180°;
when the arc-shaped X-ray array source is used, the arc-shaped X-ray array source is a single X-ray array source; wherein the arc-shaped X-ray array source comprises a plurality of ray sources distributed in an array, the arc-shaped X-ray array source surrounds the imaging object in a semi-enclosed shape, and the ray sources are arranged on a side of the panel facing the imaging object; and an arc angle range of the arc-shaped X-ray array source is 90°-180°; and
the detector is a flat-panel detector or an arc-shaped detector; the detector is configured to receive the X-ray beam emitted from the plane X-ray array source; after receiving an acquisition instruction from the data acquisition module, the detector acquires projection data of the plane X-ray array source and transmits the acquired projection data to the image reconstruction module; and the image reconstruction module reconstructs the projection data to achieve virtual rotation projection of the imaging object, and a three-dimensional digital breast tomosynthesis function is realized through a reconstruction algorithm.

2. The digital breast tomosynthesis system based on the X-ray array source according to claim 1, wherein the plane X-ray array source is located above, below, or to a side of the imaging object, and the detector is arranged on a side of the imaging object away from the plane X-ray array source.

3. The digital breast tomosynthesis system based on the X-ray array source according to claim 1, wherein when the plane X-ray array source is the flat-panel X-ray array source, the flat-panel X-ray array source comprises two ray source units, the two source units are both arranged to face the imaging object;
the two ray source units are located directly above, directly below, or on a same side of the imaging object, the two ray source units are located on a same side of the imaging object; and
the two ray source units are symmetric about a longitudinal central axis of the imaging object.

4. The digital breast tomosynthesis system based on the X-ray array source according to claim 1, wherein when the plane X-ray array source is the flat-panel X-ray array source, the flat-panel X-ray array source comprises three ray source units, the three ray source units are all arranged to face the imaging object;
the flat-panel X-ray array source comprises a first ray source unit, and a second ray source unit and a third ray source unit respectively located on both sides of the first ray source unit; the first ray source unit is located directly above, directly below, or on a same side of the imaging object, and the first ray source unit, the second ray source unit, and the third ray source unit are located on a same side of the imaging object; and
the second ray source unit and the third ray source unit are symmetric about the longitudinal central axis of the imaging object.

5. The digital breast tomosynthesis system based on the X-ray array source according to claim 1, wherein the detection module further comprises a pressing plate configured to fix the imaging object.

6. The digital breast tomosynthesis system based on the X-ray array source according to claim 1, wherein the image reconstruction module comprises a data correction unit, a data preprocessing unit, and a reconstruction unit connected in sequence;
the data correction unit is configured to perform correction processing of the projection data;
the correction processing comprises bright-field correction, dark-field correction, zero-field correction, and detector response correction; the data correction unit comprises a determination unit and a correction selection unit;
the correction selection unit comprises a phantom-based correction module and a phantom-free correction module;
the determination unit is configured to determine whether a correction phantom is present in the module;
the correction selection unit is configured to select the phantom-based correction module to correct the projection data when a correction phantom is present in the module, and the phantom-free correction module is selected to perform correction processing of the projection data when no correction phantom is present in the module;
the data preprocessing unit is configured to preprocess the corrected projection data, the preprocessing comprises beam shape correction and light intensity correction;
the reconstruction unit is configured to design a differential constraint term based on an angle of virtual rotation projection and a differential relationship equation of the detector, to optimize and solve the differential relationship equation based on the differential constraint term, and to reconstruct the preprocessed projection data to obtain an internal structure of the imaging object.

7. The digital breast tomosynthesis system based on the X-ray array source according to claim 1, wherein the plane X-ray array source further comprises a collimator configured to collimate the ray sources on the ray source units; and
the collimator is arranged inside the panel, or the collimator is arranged on a side surface of the panel facing the imaging object.

8. A digital breast tomosynthesis method based on an X-ray array source, wherein the method uses the digital breast tomosynthesis system based on the X-ray array source according to claim 1 to perform computed tomography (CT) imaging, comprising the following steps:
setting imaging parameters;
placing an imaging object on a detection platform, and setting an angle between panels of adjacent ray source units;
addressably illuminating ray sources of the ray source units under an encoding template;
acquiring projection information of all ray beams emitted by the ray sources under the encoding template through a detector to obtain projection data;
obtaining virtual rotation projection of plane X-ray array source relative to the imaging object by rearranging panel angles of the ray source units; and
designing a differential constraint term based on an angle of virtual rotation projection and a differential relationship equation of the detector, optimizing and solving the differential relationship equation based on the differential constraint term, and reconstructing the preprocessed projection data to obtain an internal structure of the imaging object.

9. The digital breast tomosynthesis method based on the X-ray array source according to claim 8, wherein the acquiring projection information of all ray beams emitted by the ray sources under the encoding template through a detector to obtain projection data comprises:
the detector sampling projection information of all ray beams emitted by the ray sources under different encoding templates to obtain projection data.

10. The digital breast tomosynthesis method based on the X-ray array source according to claim 8, wherein an acquiring process of the acquiring projection information of all ray beams emitted by the ray sources under the encoding template through a detector is expressed as:
$$b = SAf = [ \begin{matrix}0 & 1 & ⋯ & 1 & 1 \\ 1 & 1 & ⋯ & 1 & 0 \\ & & ⋱ & & \\ 0 & 0 & ⋯ & 1 & 1 \\ 1 & 1 & ⋯ & 0 & 0\end{matrix} ] [ \begin{matrix}{P}_{11} , {P}_{12} , … , {P}_{1 N} \\ {P}_{21} , {P}_{22} , … , {P}_{2 N} \\ ⋯ \\ {P}_{M 1} , {P}_{M 2} , … , {P}_{MN}\end{matrix} ] = SP$$
wherein b denotes measurement data; S denotes a sampling matrix, Sϵ [symbol] ^{K×M}; M denotes a number of light sources; l denotes that the light source at the corresponding position is illuminated in k-th measurements; P denotes a projection data matrix, that is, projection data from each point source, which is mathematically equal to Af; and A denotes a known system matrix, f denotes an image to be reconstructed, and Pᵢⱼ denotes the measurement data received by a i^{th} detector from a j^{th} light source.

11. The digital breast tomosynthesis method based on the X-ray array source according to claim 8, wherein the obtaining virtual rotation projection of plane X-ray array source relative to the imaging object by rearranging panel angles of the ray source units comprises:
cone beams being decoupled using encoding illumination to obtain single-point cone beams; and
parallel beams at different angles being then obtained by rearranging projections from the ray source units to obtain the virtual rotation projection.

12. An electronic device, comprising:
at least one processor; and,
a memory in communication connection with the at least one processor; wherein the memory stores an instruction executable by the at least one processor, and when being executed by the at least one processor, the instruction causes the at least one processor to execute the digital breast tomosynthesis method based on the X-ray array source according to claim 8.
